(19) United States
(12) Patent Application Publication
(10) Pub. No.: US 2008/0045814 A1
(43) Pub. Date: Feb. 21, 2008

(54) Method and system for the treatment of lesions

(76) Inventor: Erik Busch, Malvern, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/522,705

(22) Filed: Sep. 18, 2006

Related U.S. Application Data
(60) Provisional application No. 60/834,726, filed on Aug. 1, 2006.

(51) Int. Cl. A61B 5/00

(52) U.S. Cl. 600/301

(57) ABSTRACT

A system and method are provided for determining a medical condition of a patient. The system and method comprise providing hemodynamic monitoring, providing stenosis monitoring, retrieving hemodynamic parameters and stenosis parameters, analyzing the hemodynamic parameters and stenosis parameters, and providing a report indicative of an integrated analysis of the hemodynamic parameters and the stenosis parameters.

### BACKGROUND

1. Field of the Invention

The present invention is generally directed to medical diagnostics. More particularly, embodiments of the present invention are directed to a method and system for providing integrated multimodality functionality in an angiographic system.

2. Background of the Invention

In the field of medical diagnostics, integrating various systems to provide a multimodality system is the current trend due to economics and efficiency. This can be seen in the medical imaging field, for example. Various systems exist such as SPECT, PET and CT. Each system provides an image of a patient's tissue, bone or blood vessels.

Previously, patients would have to use separate systems in order for medical personnel to acquire images of interest comprising blood vessels, bone and tissue. This was inconvenient for the patient because the patient had to change machines and the medical personnel had to look at different films showing the different areas of interest. However, the use of multimodality systems such as SPECT/PET and SPECT/CT allow medical personnel to view blood vessels, bone and tissue on the same film. This results in medical personnel analyzing information in an integrated setting by taking all three features into account simultaneously rather than looking at each area of interest separately in an isolated environment.

In contrast, in the treatment of lesions also known as stenosis, the systems also have a numerical focus and not just an image focus. Therefore, there is probably a greater need to create synergy by integrating various systems. Currently, systems exist that measure hemodynamic parameters and the degree of stenosis (stenosis parameters). However, stenosis parameters and hemodynamic parameters are measured and communicated using separate systems. That is, medical personnel determine the diagnosis based on the measurement of separate systems. The analysis of the detected parameters is determined separately and manually by the medical personnel.

The following problems may occur—the manual analysis may result in the treatment of lesions without considering the hemodynamics in the blood vessel. This may result in suboptimal results in patient care and safety, and additional costs without an improvement in the patient's situation.

In addition, the outcome of the analysis is subject to the training and experience of the medical personnel. Medical personnel having little experience should have their analysis reviewed by someone with greater experience. Unfortunately, this is not always done due to budget concerns, carelessness and lack of personnel.

Thus, there is a need for a method and system that provides an integrated multimodality analysis of stenosis and hemodynamic parameters.

### SUMMARY OF THE INVENTION

It is therefore an object of the present invention to provide a system and method where an integrated multimodality analysis of stenosis and hemodynamic parameters is performed. Preferably, this is accomplished in a manner in which a graphical, numerical or audible output is provided indicating a patient's condition.

A system and method are provided for determining a medical condition of a patient. The system and method comprise providing hemodynamic monitoring, providing stenosis monitoring, retrieving hemodynamic parameters and stenosis parameters, analyzing the hemodynamic parameters and stenosis parameters, and providing a report indicative of an integrated analysis of the hemodynamic parameters and the stenosis parameters.

In an aspect of the present invention, the report comprises a graph. In another aspect of the present invention, the graph comprises at least two regions indicative of a Normal condition, a Critical condition, and an Acute Danger condition.

In a further aspect of the invention, the graph is color coded and each one of the regions has a different color.

In still a further aspect of the present invention, there is a transitionary area between each of the regions.

### BRIEF DESCRIPTION OF THE DRAWINGS

A wide array of potential embodiments can be better understood through the following detailed description and the accompanying drawings in which:

FIG. 1A is a diagram illustrating an exemplary medical imaging device in accordance with an embodiment of the present invention;

FIG. 1B is a block diagram illustrating exemplary apparatuses for providing physiological parameters and hemodynamic parameters in accordance with an embodiment of the present invention;

FIG. 2 is a flow chart illustrating a process for monitoring and analyzing the physiological parameters and hemodynamic parameters in accordance with an embodiment of the present invention;

FIG. 3 is a block diagram of a computer for optimizing user performance in accordance with an embodiment of the present invention; and

FIG. 4 is a graph illustrating exemplary regions for categorizing a patient's medical condition based on a combined analysis of the stenosis parameters and hemodynamic parameters in accordance with an embodiment of the present invention.

In the drawings, the same or similar elements are denoted by the same reference numerals even though they are depicted in different drawings.

### DETAILED DESCRIPTION OF EXEMPLARY EMBODIMENTS

As required, detailed embodiments of the present inventions are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

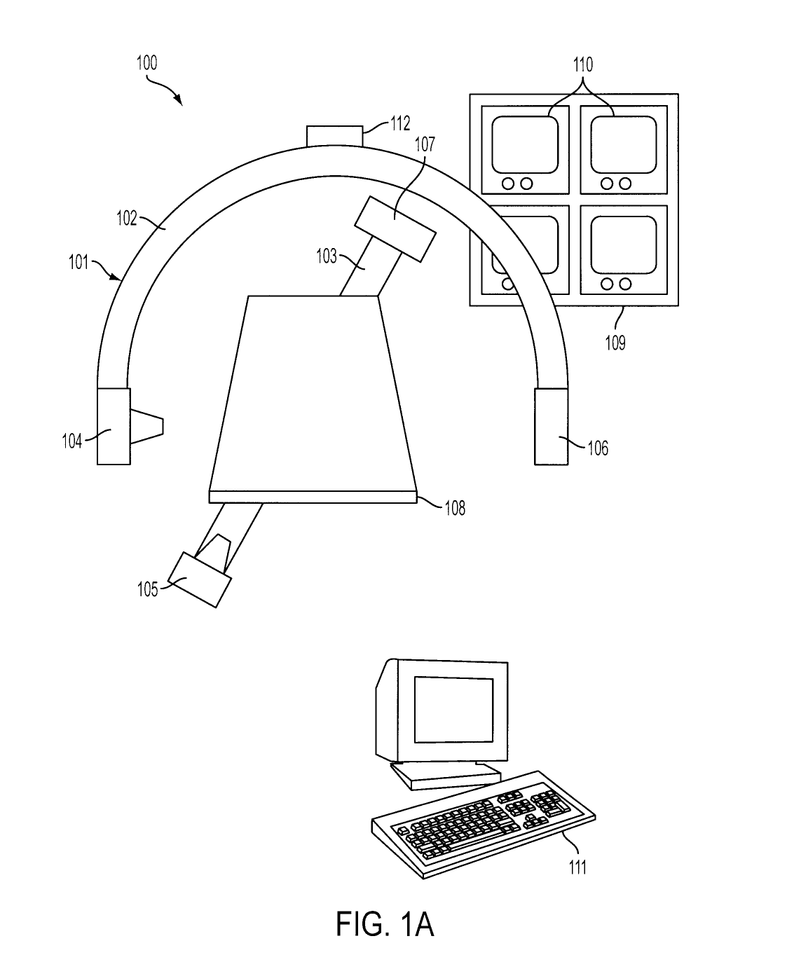

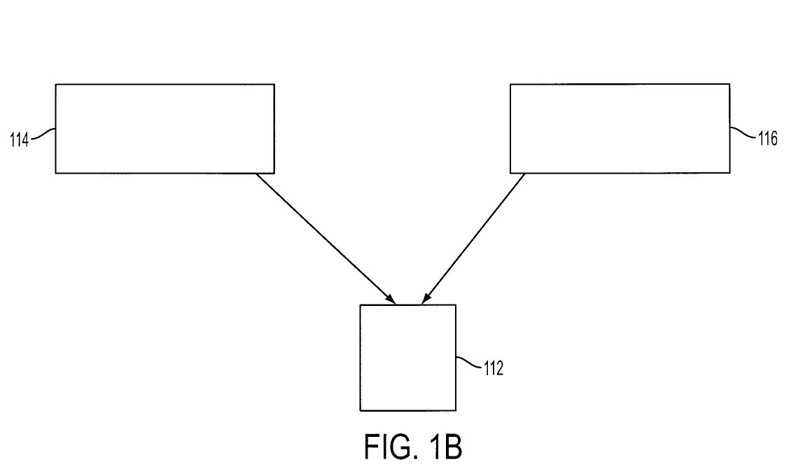

FIG. 1 is a diagram illustrating an exemplary medical imaging device 100 in accordance with an embodiment of the present invention. The medical imaging device comprises two C-arms 102 and 103, at the respective ends of which X-ray emitters 104 and 105 as well as X-ray detectors 106 and 107 situated opposite to each other in a known fashion. For example, flat detectors are installed.

It should be appreciated by those skilled in the art that the present invention can be performed without C-arms without departing from the scope of the present invention. For example, any medical imaging device having an emitter and detector can be used e.g., MRI.

In addition, the medical imaging device 100 is provided with a patient examination table 108. For observation of the examination, a monitor support or monitor bank 109 is provided, in this example, comprising four monitors 110. However, a conventional medical imaging device 100 comprises at least one display.

An operating console 111 is located in an adjacent control room for communication with the system for the purpose of controlling the C-arms 102 and 103 and/or X-ray emitters 104 and 105 as well as X-ray detectors 106 and 107, image generation and image processing. Typically, an operating console 111 in the control room is provided with at least two monitors for biplane systems. It should be appreciated by those skilled in the art that other configurations are possible.

The C-arms 102 and 103 can be ceiling mounted and/or floor mounted. A combination of floor and ceiling mounted C-arms allow for adaptable positioning of the medical imaging device and fast programmable movement. It also allows peripheral examinations to be performed without repositioning a patient. The medical imaging device 100 comprises a biplane angiography system with flexible and ergonomic architecture that also allows medical personnel to adapt the medical imaging device 100 configuration for cardiologic and stenosis applications.

In accordance with an embodiment of the present invention, a processor 112 is provided for the medical imaging device 100. The processor 112 includes memory comprising different procedures to implement after analyzing the stenosis parameters and the hemodynamic parameters.

In accordance with an embodiment of the present invention, the medical imaging device 100 is equipped with an apparatus for monitoring and reading stenosis parameters and hemodynamic parameters. Table 1 illustrates some exemplary stenosis parameters without normal values shown. The normal values vary based on age and sex of the subject. Those skilled in the art are aware of the values.

TABLE 1
Stenosis Parameters

Minimum Luminal Diameter
% Diameter Stenosis
Reference Diameter
Length Stenotic Segment
Area at MLD Densitometry
Area at MLD Circular
% Area Stenosis at MLD Densitometry
% Area Stenosis at MLD Circular
Reference Area
Volume Stenotic Segment
Plaque Area
Plaque Volume

The imaging device 100 employs conventional invasive and noninvasive methods to determine stenosis parameters and hemodynamic parameters as is known to those skilled in the art. For example, the medical imaging device 100 can perform the functions of various devices and acquire stenosis parameters and hemodynamic parameters either separately or together.

Similarly, there are different techniques for acquiring hemodynamic parameters that are known to those skilled in the art. The accuracy of the reading, again, depends on the experience and training of the person interpreting the parameters to determine a root cause of a deviation in readings. Hemodynamic parameters outside the normal range can indicate a myriad of problems. For example, symptoms can be anything from cardiac dysfunction, pulmonary edema, rales, increased jugular vein size, pulmonary edema, complete cardiovascular collapse, and profound shock. Symptoms can also include weakness, pallor, confusion, cold clammy skin, diminished or absent pulses, cardiac arrhythmias, low arterial blood pressure, murmurs and decreased cardiac output. Table 2 illustrates some exemplary hemodynamic parameters.

TABLE 2

| Hemodynamic Parameters | Abbreviations | Normal Values | |
|---|---|---|---|
| Mean Arterial Pressure | MAP | 70–90 | mmHg |
| Right Artrial Pressure | RAP | 2–6 | mmHg |
| Central Venous Pressure | CVP | 2–8 | mmHg |
| Pulmonary Artery Systolic | PAS | 20–30 | mmHg |
| Pressure | | | |
| Pulmonary Diastolic | PAD | 6–12 | mmHg |
| Systolic Pressure | | | |
| Pulmonary Artery Mean | PAM | 10–15 | mmHg |
| Pressure | | | |
| Pulmonary Artery Wedge | PAWP, Wedge | 8–12 | mmHg |
| Pressure | | | |
| Cardiac Output | CO | 4–8 | L/min |
| Cardiac Index | CI | 2.5–4 | L/min |
| Stroke Volume | SV | 60–130 | ml |
| Stroke Volume Index | SVI | 40–50 | ml/m2 |
| Systemic Vascular | SVR | 800–1200 | dynes |
| Resistance | | | |
| Systemic Vascular | SVRI | 2000–2400 | dynes |
| Resistance Index | | | |
| Pulmonary Vascular | PVR | 150–300 | dynes |
| Resistance | | | |

It should be appreciated by those skilled in the art that some of the parameters may be calculated based on other available parameters. For example, cardiac output can be measured using the formula CO=SV*HR

Where CO=cardiac output (liter/min)

SV=stroke volume, the volume of blood ejected from the heart due to contraction of the left ventricular.

HR=hear rate, number of heart beats per minute.

Preferably, both the stenosis parameters and the hemodynamic parameters should be analyzed together. In accordance with an embodiment of the present invention, processor 112 analyzes the stenosis parameters and the hemodynamic parameters. Values that are outside the normal range indicate a problem. More weight can be given to parameters that are considered critical. Critical parameters are well known by those skilled in the art.

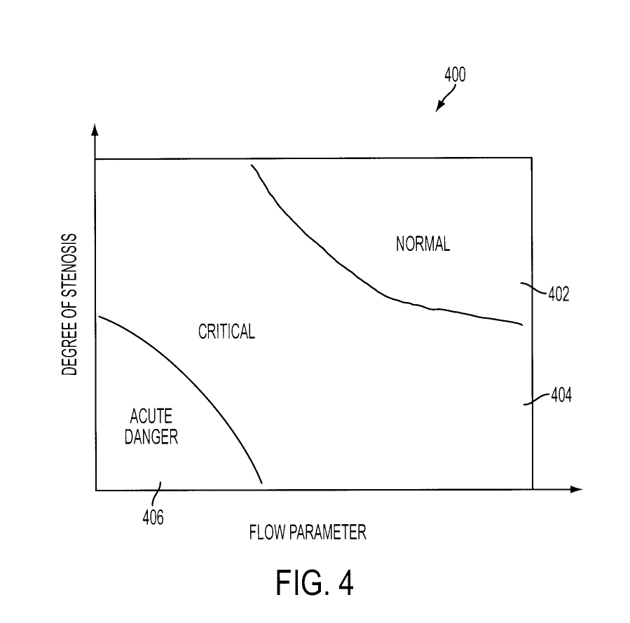

After the stenosis parameters and the hemodynamic parameters are analyzed a determination is made as to whether the readings fall into one of at least two categories or regions as shown in the graph of FIG. 4. It should be appreciated by those skilled in the art that the number of regions illustrated is exemplary. More or less than three regions can be used without departing from the scope of the present invention.

Returning to FIG. 4, an asterisk or any suitable symbol can be used to indicate which category or region the patient's condition places the patient. Graph 400 indicates the degree of stenosis on the y-axis and the flow parameter on the x-axis. The Normal region 402 indicates that all or substantially all the parameters came back within the normal range. The Critical region 404 indicates that a problem has been detected, and the patient had a few parameters outside the normal range. A printout will be provided of the values outside the normal range. These values can be highlighted, in color, bold and/or in a separate grouping from the parameters that came within the normal range. The Acute Danger region 406 indicates that a major problem has been detected concerning the patient's condition. For instance, anyone of the parameters that are defined as critical may have been exceeded or a number of the noncritical parameters may have been outside of the normal range. Again, these values can be highlighted, in color, bold and/or in a separate grouping from the parameters that came within the normal range.

Graph 400 may be color coded. For example, the normal region may be shown as blue, the critical region shown as orange and the acute danger region shown as red. It should be appreciated by those skilled in the art that the present invention is not limited to the disclosed colors. Other colors may be used without departing from the scope of the present invention.

The medical imaging device 100 can recommend interventional procedures to the medical personnel. For example medication and surgical procedures can be displayed and/or printed out. The patient's medical history can also be viewed and compared. For instance, based on the graph 400, specific treatment for the patient can be recommended. For example, stent size, length and material and deployment pressure may also be recommended.

It should be appreciated by those skilled in the art that the graph 400 may also comprise a text based report disclosing parameters that fall outside the norm and the problem the patient may have. In addition, a numerical report can also be provided listing the stenosis parameters and the hemodynamic parameters. In an embodiment of the present invention, the stenosis parameters that fall outside the range can be viewed as a graph separate from graph 400 allowing the medical personnel to review the measurements in question manually.

In accordance with a further embodiment of the present invention, a third modality may comprise an x-ray. For example, medical personnel may detect a problem with a patient using graph 400 in accordance with an embodiment of the present invention. Medical personnel may then take an x-ray of the patient. Based on the graph 400 and textual information, the medical imaging device 100 can focus on the problem areas e.g., capture an image of the pulmonary area if the stenosis parameters and/or the hemodynamic parameters showed that this area of the heart had a problem. Thus, cost savings can be envisioned by comparing the stenosis parameters and hemodynamic parameters, providing a graphical or textual detailed analysis of the problem and determining whether to take an x-ray based on the results of the analysis.

It should be appreciated by those skilled in the art that the present invention can be practiced using distinct apparatuses. For example an apparatus for monitoring stenosis parameters 114 and an apparatus for monitoring hemodynamic parameters 116 may be connected to an apparatus having processor 112 as shown in FIG. 1B. Processor 112 can then process the parameters and provide graph 400 and/or textual information. In addition, processor 112 can also be connected to an image processing device to allow the device to focus on the detected problem area. The connection to a medical imaging device may be practiced via a portable memory device or through a network such as a local area network, a wide area network or the Internet. Thus, portability is added by taking portable memory from processor 112 and connecting it to a medical imaging device or having the patient go to another room or location and remotely retrieving the information prior to or after taking an x-ray.

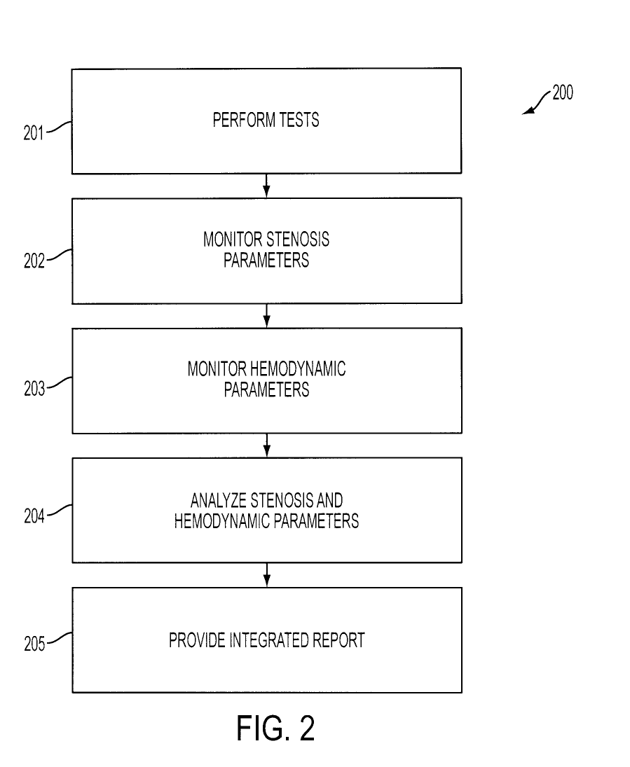

FIG. 2 is a flow chart illustrating a process 200 for measuring and analyzing the usage of dose reduction features in accordance with an embodiment of the present invention.

At steps 201, 202 and 203, tests are performed on a patient. The tests may comprise at least one of an x-ray, the monitoring of stenosis parameters and hemodynamic parameters. The test for the stenosis parameters and hemodynamic parameters may be performed using a separate apparatus or an integrated apparatus.

At step 204, the stenosis parameters and hemodynamic parameters are analyzed by processor 112. Patterns are determined and/or problem areas are detected. Since a processor is analyzing the information a much more accurate analysis can be provided compared to a human analysis. Minor details in a graph, for example, can be analyzed to determine if there is a pattern that can indicate heart disease.

At step 205, an output is provided by processor 112. The output may comprise a report on a screen, a printout or an output to a portable memory device. The output may comprise a report such as graph 400 which indicates a Normal, Critical, or Acute Danger state of the patient based on a combined analysis of the stenosis parameters and hemodynamic parameters. The different categories or regions may be color coded to provide a visual distinction among the regions.

In a further embodiment of the present invention, a buffer color can be provided around the transition areas. For example, rather than having a clear transition between the Normal and Critical regions, there can be a region of a different color than the two connecting regions. This can allow medical personnel to issue a warning to the patient that their condition may not have reached the critical stage but the patient's condition is close to being critical.

The report can also provide a textual indication of the stenosis parameters and hemodynamic parameters that were outside the norm to allow the medical personnel to review these parameters or all the parameters.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

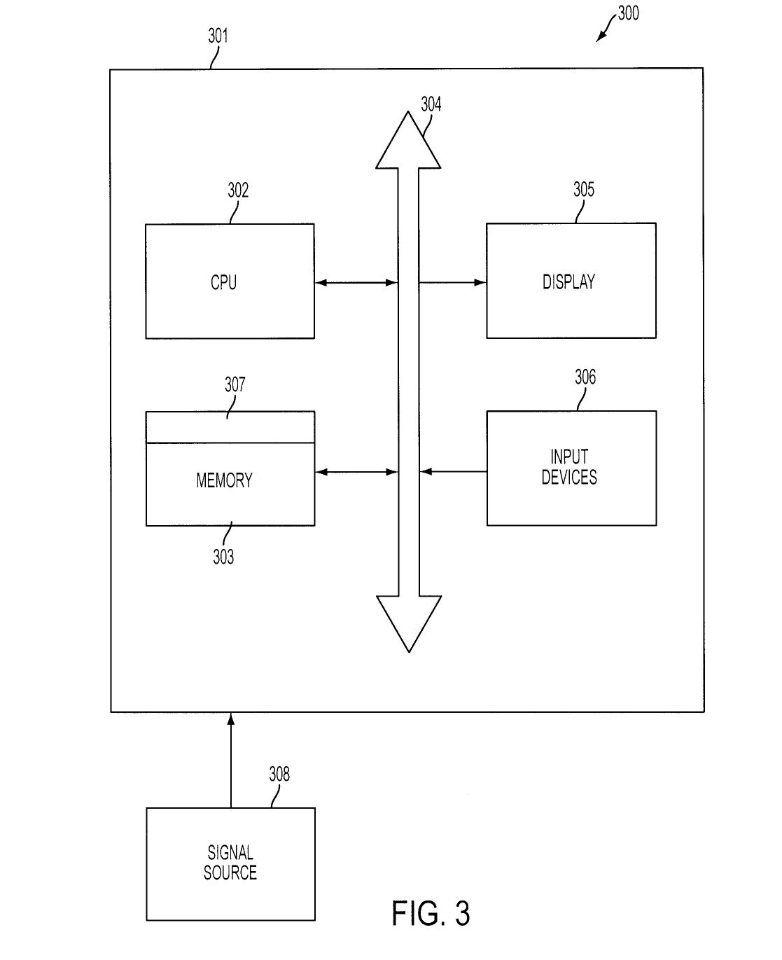

Referring now to FIG. 3, according to an embodiment of the present invention, a computer system 301 for implementing the present invention can comprise, inter alia, a central processing unit (CPU) 302, a memory 303 and an input/output (I/O) interface 304. The computer system 301 is generally coupled through the I/O interface 304 to a display 305 and various input devices 306 such as a mouse and a keyboard. The support circuits can include circuits such as cache, power supplies, clock circuits, and a communication bus. The memory 303 can include random access memory (RAM), read only memory (ROM), disk drive, tape drive, etc., or a combinations thereof. The present invention can be implemented as a routine 307 that is stored in memory 303 and executed by the CPU 302 to process the signal from the signal source 308. As such, the computer system 301 is a general purpose computer system that becomes a specific purpose computer system when executing the routine 307 of the present invention.

The computer system 301 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

It is to be further understood that, because some of the constituent system components and method steps depicted in the accompanying figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A method for determining a medical condition of a patient, the method comprising:
providing hemodynamic monitoring;
providing stenosis monitoring;
retrieving hemodynamic parameters and stenosis parameters;
analyzing the hemodynamic parameters and stenosis parameters; and
providing a report indicative of an integrated analysis of the hemodynamic parameters and the stenosis parameters.

2. The method of claim 1, wherein said step of providing a report further comprises:
providing a characteristic of blood flow.

3. The method of claim 1, wherein said step of providing a report further comprises:
indicating if the medical condition of the patient falls within one of at least two regions.

4. The method of claim 3, wherein the regions comprises:
at least one of a normal indication, a critical indication and an acute danger indication.

5. The method of claim 4, wherein the report comprises a graph.

6. The method of claim 5, wherein the graph is color coded.

7. The method of claim 3, wherein each one of the at least two regions comprises a different color.

8. The method of claim 7, wherein each one of the at least two regions is separated by a transition area having a different color from adjacent regions.

9. The method of claim 1, wherein the report provides a text output of the hemodynamic parameters and the stenosis parameters.

10. The method of claim 1, wherein the report provides a text output of the hemodynamic parameters and a graphical output of the stenosis parameters.

11. The method of claim 1, further comprising:
taking an x-ray of the patient based on a result of the report.

12. A system for determining a medical condition of a patient, comprising:
a first apparatus for providing hemodynamic monitoring;
a second apparatus for providing stenosis monitoring;
a third apparatus for retrieving hemodynamic parameters and stenosis parameters from the first apparatus and the second apparatus, analyzing the hemodynamic parameters and stenosis parameters, and
providing an output indicative of an integrated analysis of the hemodynamic parameters and the stenosis parameters.

13. The system of claim 12, wherein results of said output indicates heart disease.

14. The system of claim 12, wherein said third apparatus provides a characteristic of blood flow.

15. The system of claim 12, wherein said output comprises a graph.

16. The system of claim 15, wherein said graph comprises at least two regions providing at least one of a normal indication, a critical indication and an acute danger indication.

17. The system of claim 15, wherein the graph further comprises transitionary regions between each of the at least two regions.

18. The system of claim 15, wherein the graph is color coded.

19. The system of claim 16, wherein each one of the at least two regions comprises a different color.

20. The system of claim 12, wherein the output provides at least one of a textual output of the hemodynamic parameters and the stenosis parameters and a graphical output indicating a condition of the patient based on the analysis.

21. The system of claim 12, wherein the output is saved onto at least one of a stationary and a portable memory device.

22. The system of claim 12, further comprising:
a fourth apparatus for providing a medical image based on the output of said third apparatus.
